(19) United States
(12) Patent Application Publication
(10) Pub. No.: US 2015/0174040 A1
(43) Pub. Date: Jun. 25, 2015

(54) ANTIPERSPIRANT COMPOSITION WITH NON-IONIC POLYMERS

(71) Applicant: The Dial Corporation, Scottsdale, AZ (US)

(72) Inventors: Jennifer Harris, Phoenix, AZ (US); Debra K. Butterworth, Scottsdale, AZ (US)

(73) Assignee: The Dial Corporation, Scottsdale, AZ (US)

(21) Appl. No.: 14/139,912

(22) Filed: Dec. 24, 2013

(51) Int. Cl. A61K 8/73; A61Q 15/00; A61K 8/28

(52) U.S. Cl. CPC A61K 8/731; A61K 8/28; A61K 8/732; A61Q 15/00

(57) ABSTRACT

Materials and apparatus are provided that include non-ionic polymers. An antiperspirant composition with non-ionic polymers includes an active antiperspirant ingredient disposed within the antiperspirant composition. The antiperspirant composition further includes a non-ionic modified cellulose polymer disposed within the antiperspirant composition. The non-ionic modified cellulose polymer forms between 0.01 to 5.0 weight percent of the antiperspirant composition. The antiperspirant composition further includes a non-ionic calcium salt disposed within the antiperspirant composition. The non-ionic calcium salt forms between 0.01 to 5.0 weight percent of the antiperspirant composition.

### FIELD OF THE INVENTION

The present invention generally relates to a composition to reduce a sensation of wetness during application of an antiperspirant, and more particularly relates to an antiperspirant composition with non-ionic polymers.

### BACKGROUND OF THE INVENTION

Antiperspirants are personal care products that are used to prevent or reduce perspiration and odors resulting from perspiration, particularly perspiration from a person's underarm. Such antiperspirants may be applied directly to skin, for example to a person's underarm. The “feel” of an antiperspirant, including the “feel” during application of an antiperspirant may contribute to a person's satisfaction with a particular antiperspirant.

Accordingly, it is desirable to have an antiperspirant composition that results in satisfactory application aesthetics. Additionally, it is desirable to have an antiperspirant composition that maintains stability during application. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An antiperspirant composition with non-ionic polymers includes an active antiperspirant ingredient disposed within the antiperspirant composition. The antiperspirant composition also includes a non-ionic modified cellulose polymer disposed within the antiperspirant composition. The non-ionic modified cellulose polymer forming between 0.01 to 5.0 weight percent of the antiperspirant composition. The antiperspirant composition also includes a non-ionic calcium salt disposed within the antiperspirant composition. The non-ionic calcium salt forming between 0.01 to 5.0 weight percent of the antiperspirant composition.

An antiperspirant product with non-ionic polymers includes a container and an antiperspirant composition housed within the container. The antiperspirant composition includes an active antiperspirant ingredient. The antiperspirant composition also includes a non-ionic modified cellulose polymer forming between 0.01 to 5.0 weight percent of the antiperspirant composition. The antiperspirant composition also includes a non-ionic calcium salt forming between 0.01 to 5.0 weight percent of the antiperspirant composition.

An antiperspirant composition with non-ionic polymers includes an active antiperspirant ingredient disposed within the antiperspirant composition. The antiperspirant composition also includes hydroxyethylcellulose forming between 0.01 to 5.0 weight percent of the antiperspirant composition. The hydroxyethylcellulose is configured to bind ingredients of the antiperspirant composition. The antiperspirant composition also includes modified corn starch forming between 0.01 to 5.0 weight percent of the antiperspirant composition. The modified corn starch is configured to bind ingredients of the antiperspirant composition.

### BRIEF DESCRIPTION OF THE DRAWINGS

The present invention will hereinafter be described in conjunction with the following drawing figures, wherein like numerals denote like elements, and

FIG. 1 is a diagram of an example of a container for dispensing an antiperspirant composition with non-ionic polymers according to the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As described above, perspiration is a common occurrence in most individual's lives. Perspiration itself and odors emanating from the perspiration can be uncomfortable, unsightly, and embarrassing. To combat perspiration, an individual may use an antiperspirant substance to reduce the perspiration itself and odors emanating from the perspiration. Such antiperspirant substances may be different types of substances that are applied to the skin, for example a person's underarm. During application of the antiperspirant and while the antiperspirant is in contact with the skin the feel, or aesthetics, of the antiperspirant may contribute to a satisfactory consumer experience. For example, a wet feeling, or sticky feeling during application of an antiperspirant may contribute to a less than satisfactory consumer experience.

Antiperspirants may come in various forms. For example, some antiperspirants are invisible solid stick antiperspirants, clear gel antiperspirants, and roll-on antiperspirants. Some of these antiperspirant types may exhibit application aesthetics that lead to a less than satisfactory user experience. For example, some antiperspirant substances may feel “sticky” and may be difficult to apply. Moreover, various types of antiperspirant may leave residues on the skin surface during use.

Accordingly, the principles described herein may provide a composition that exhibits desirable application aesthetics. Desirable application aesthetics may include soft and silky application that does not leave a wet feeling during, or after, application. The antiperspirant composition may include a number of non-ionic polymers for binding other ingredients disposed within the antiperspirant composition. This binding may increase the feel of the antiperspirant composition while in contact with the skin and more particularly during application of the antiperspirant composition. For example, the antiperspirant composition with non-ionic polymers may reduce a feeling of wetness of the antiperspirant composition during application.

The presence of non-ionic polymers may be a unique aspect of the invention as general practice in the field does not rely on such polymers, in particular hydroxyethylcellulose and modified corn starch, to act as binders in an antiperspirant composition. Accordingly, the presence of non-ionic polymers in the antiperspirant composition may be beneficial in that it improves the application and contact aesthetics of the antiperspirant composition.

Accordingly, the antiperspirant composition disclosed herein may provide improved aesthetics, matrix stability, all while maintaining antiperspirant efficacy to leave the consumer with an overall satisfactory experience.

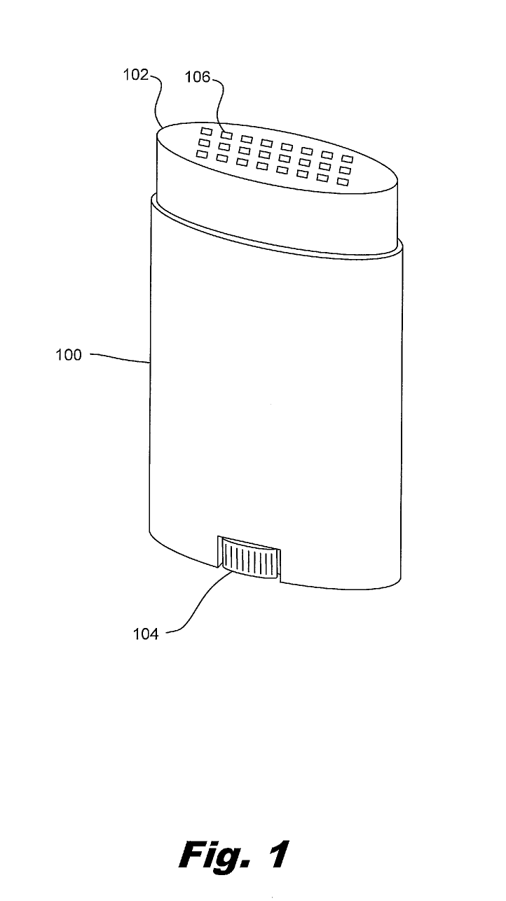

Turning now to the figures, FIG. 1 is a diagram of an example of a container (100) for dispensing an antiperspirant composition with non-ionic polymers according to the principles described herein. In this example, the antiperspirant composition may be held within a container (100) that has a top surface (102) that allows the antiperspirant composition to be pushed out of the container (100) and onto a surface. For example, the antiperspirant composition may pass through a grate (106) on the top surface (102) to extend beyond the top surface (102) of the container (100) and be applied to skin. For example, the antiperspirant composition may be applied to an underarm. The container (100) may also include a dispenser to dispense the antiperspirant composition. For example, the antiperspirant composition can be pushed out of the grate (106) by rotating a dial (104), or a knob, positioned at the bottom of the container (100). As the dial (104) is rotated, a platform internal to the container (100) moves up and pushes the antiperspirant composition with it. As will be described in detail below, the container (100) may contain a grate (106) through which the antiperspirant composition is pushed. In this example, the antiperspirant composition may be a semi-solid, or soft solid, antiperspirant composition.

Any appropriate type of container (100) may be used to hold and apply the antiperspirant composition to skin. For example, a container (100) with an oval cross section may be used to hold the antiperspirant composition (100). Also, a container (100) with a more cylindrical cross section may be used in some examples. The container (100) may have any appropriate shape in accordance with the principles described herein. In some examples, the container (100) may include a protective cover.

During use, a top of the container (100) may be removed and the antiperspirant composition exposed. Via the container (100), a user may apply the antiperspirant composition to skin by rubbing the antiperspirant composition across the skin.

In some examples, the antiperspirant composition may be a semi-solid antiperspirant composition. A semi-solid antiperspirant composition may be an antiperspirant composition that is not hard. The antiperspirant composition may not have a form when not included in a container. The antiperspirant composition may be a soft solid. In some examples, a semi-solid antiperspirant composition may be any antiperspirant composition that is configured to be pushed through a grate (106) of a container (100). For example, as described above, a container (100) may include a dial (104) that upon rotation pushes the antiperspirant composition up. In this example, the rotation of the dial (104) may push the antiperspirant composition through the grate (106) to be distributed to a skin surface such as a person's underarm.

The antiperspirant composition may include an active antiperspirant ingredient. The active antiperspirant ingredient may be any ingredient that may reduce perspiration, perspiration odors, wetness resulting from perspiration, or combinations thereof. In some examples, the active antiperspirant ingredient may be a salt that reduces perspiration by diffusing through the sweat ducts of sweat glands. The salts may then combine with proteins to plug sweat ducts.

Examples of active antiperspirant ingredients include astringent water-soluble inorganic and organic salts of aluminum, zirconium and zinc or any mixtures of these salts. Examples of active antiperspirant ingredients include, aluminum zirconium techlorohydrex, aluminum halides, aluminum chlorohydrates, aluminum hydroxyhalides, zirconyl oxyhalides, zirconyl hydroxyhalides, aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, and combinations thereof.

While the above examples have been described with reference to specific types of active antiperspirant ingredients, any appropriate antiperspirant ingredient may be used in accordance with the principles described herein. For example, the active antiperspirant ingredient may be used to prevent perspiration and perspiration odors, inhibit the formation of perspiration and perspiration odors, or otherwise contribute to reducing perspiration. Further, the active antiperspirant ingredients may include multiple types of active antiperspirant ingredients that reduce perspiration and perspiration odors. In such examples, each of the active antiperspirant ingredients may perform different functions, perform overlapping functions, perform the same functions, or combinations thereof.

The antiperspirant composition may include the active antiperspirant ingredient in an amount that provides an antiperspirant effect. For example, the active antiperspirant ingredient may form between 20.0 and 30.0 weight percent of the antiperspirant composition, such as 25.478 weight percent.

The antiperspirant composition may also include an active carrier that delivers the active antiperspirant ingredient throughout the antiperspirant composition (100). A carrier may be any compound or mixture configured to deliver other ingredients. Accordingly, the antiperspirant composition may include any compound or mixture that is configured to deliver other ingredients to the skin. A specific example of an active carrier is cyclopentasiloxane. Other examples of active carriers include cyclic silicones, linear silicones, or combinations thereof. The antiperspirant composition may include an active carrier in an amount to distribute the active antiperspirant ingredient throughout the antiperspirant composition. For example, the active carrier may form between 30.0 and 35.0 weight percent of the antiperspirant composition (100), such as 32.9573 weight percent.

As will be described below, the active antiperspirant ingredient and the active carrier may form part of a premix that may be combined, and then later mixed with other ingredients to form a semi-solid antiperspirant composition. The premix of the active antiperspirant ingredient and the active carrier may form between 50.0 and 60.0 weight percent of the antiperspirant composition, such as 58.435 weight percent.

The antiperspirant composition may include a non-ionic modified cellulose polymer. The non-ionic modified cellulose polymer may bind to ingredients that lead to a feeling of wetness. In some examples, the ingredients that may lead to a feeling of wetness include silicones that may be included in an antiperspirant composition. As such, the non-ionic modified cellulose polymer may reduce a sensation of wetness during application of the antiperspirant composition. In one example, the non-ionic modified cellulose polymer may absorb these ingredients that lead to a feeling of wetness. In this example, the non-ionic modified cellulose polymer may reduce a sensation of wetness as the ingredients that cause such a sensation are absorbed by the modified cellulose polymer. The modified cellulose polymer may contribute to a more stable antiperspirant composition matrix. With a more stable antiperspirant composition matrix, a carrier fluid of the antiperspirant composition may be more tightly bound in the formula so as to not produce perceived wetness during dispensing and application. Additionally, this may reduce a visual separation of the carrier fluid from the antiperspirant composition matrix. This separation may be referred to as syneresis.

As the modified cellulose polymer is non-ionic, it may be stable in compositions featuring active antiperspirant ingredients with high salt concentrations. Accordingly, the non-ionic modified cellulose polymer may provide improved smoothness and silkiness in the antiperspirant composition while reducing a perception of wetness on a skin surface.

In some examples, the modified cellulose polymer may be a hydrophilic film-forming ingredient. A film-forming ingredient may refer to any compound that leaves a film on a skin surface. A film-forming modified cellulose polymer may leave a smooth feel on the skin, thus increasing the application aesthetics and the contact aesthetics of the antiperspirant composition.

In one example, the modified cellulose polymer may be a pseudo-plastic. That is, the modified cellulose polymer may be configured to retain its form in the presence of a surface stress. For example, as described above, in some examples, the antiperspirant composition may be pushed through a grate (106) of a container (100). Doing so may put stress on the antiperspirant composition, which may contribute to a perceived sensation of wetness during application. In some examples, the perceived sensation of wetness may be due to a syneresis, or separation of the active carrier from the antiperspirant composition matrix. Accordingly, a pseudo-plastic modified cellulose polymer binder may be beneficial in that the antiperspirant composition maintains its form even after the stress of passing through the grate (106) in the container (100) or in the presence of other surface stress such as during application. The reduced stress may allow the antiperspirant composition to retain its form, thus reducing the sensation of wetness that may result from a breakdown of the ingredients within the antiperspirant composition in the presence of stress imposed by the grate (106) or during application.

In one specific example, the modified cellulose polymer may be hydroxyethylcellulose. The modified cellulose polymer may form between 0.01 and 5.0 weight percent of the antiperspirant composition, such as between 0.01 and 1.0 percent. In some examples, the antiperspirant composition may include an amount of the modified cellulose polymer to achieve a binding effect.

The antiperspirant composition may include a non-ionic calcium salt. The non-ionic calcium salt may bind to ingredients that lead to a feeling of wetness, such as silicones that may be present in the antiperspirant composition. As such, the non-ionic calcium salt may reduce a sensation of wetness during application of the antiperspirant composition. In one example, the non-ionic calcium salt may absorb these other ingredients that lead to a feeling of wetness. In this example, the non-ionic calcium salt may reduce a sensation of wetness as the ingredients that cause such a sensation are absorbed by the non-ionic calcium salt. The non-ionic calcium salt may create a more stable antiperspirant composition matrix. With a more stable antiperspirant composition matrix a carrier fluid of the antiperspirant composition may be more tightly bound in the formula so as to not produce perceived wetness during dispensing and application. Additionally, this may reduce a visual separation of the carrier fluid from the antiperspirant composition matrix. This separation may be referred to as syneresis.

As the calcium salt is non-ionic, it may be stable in compositions featuring active antiperspirant ingredients with high salt concentrations. Accordingly, the non-ionic calcium salt may provide improved smoothness and silkiness in the antiperspirant composition while reducing a perception of wetness on a skin surface.

In some examples, the calcium salt may be a hydrophilic film-forming ingredient. A film-forming ingredient may refer to any compound that leaves a film on a skin surface. A film-forming calcium salt may leave a smooth feel on the skin, thus increasing the application aesthetics and the contact aesthetics of the antiperspirant composition.

In one specific example, the calcium salt may be modified corn starch. Modified corn starch may be a calcium salt of the ester formed from a reaction of 3-(dodecenyl)dihidri-2,5 furandione and corn starch. The modified cellulose polymer may form between 0.01 and 5.0 weight percent of the antiperspirant composition, such as between 0.01 and 1.0 weight percent. In some examples, the antiperspirant composition may include an amount of the modified cellulose polymer to achieve a binding effect.

A combination of a non-ionic modified cellulose polymer such as hydroxyethylcellulose and a non-ionic calcium salt such as modified corn starch may be a unique aspect of the antiperspirant composition as general practice in the field is to rely on other combinations of binders or absorbents. Additionally, including the non-ionic modified cellulose polymer and non-ionic calcium salt in amounts ranging from 0.01 to 5.0 weight percent of the antiperspirant composition may be a unique aspect as general practice in the field is to rely on greater amounts of binders or absorbents.

The use of a non-ionic modified cellulose polymer and a non-ionic calcium salt may be beneficial in that it may reduce perceived wetness resulting from the stress on an antiperspirant composition as it passes through a grate (106) in a top surface (104), or stress resulting from application of the antiperspirant composition. For example, as described above, when passing a semi-solid through a grate (106) during application of an antiperspirant composition, the grate (106) may exert stress on the antiperspirant matrix which may degrade the antiperspirant composition and may contribute to a sensation of wetness during application. Similarly, a stress may be exerted as the antiperspirant composition is rubbed across a skin surface during application. Accordingly, the antiperspirant composition as described herein, by using a unique combination of a non-ionic modified cellulose polymer and a non-ionic calcium salt that may bind with other ingredients, absorb other ingredients, act as a pseudo-plastic or combinations thereof, may reduce the effects of stress on the antiperspirant composition resulting in less degradation of the antiperspirant composition matrix. Accordingly, the application aesthetics of the antiperspirant composition may be maintained. More specifically, the sensation of wetness during application may be reduced as the strength of the antiperspirant composition is maintained in the presence of stress exerted by the grate (106) of a container (100) or during application.

In addition to the ingredients mentioned above, the antiperspirant composition may include other ingredients to improve the application aesthetics, contact aesthetics, or other characteristics of the antiperspirant composition. For example, the antiperspirant composition may include a structurant. A structurant may be any compound or mixture configured to give shape, or structure to the antiperspirant composition. Accordingly, the antiperspirant composition may include any compound or mixture that is configured to give shape, or structure to the antiperspirant composition. Examples of structurants may include stearyl alcohol, soy wax, cetyl alcohol, polyethylene or combinations thereof.

The antiperspirant composition may include an emollient. An emollient may refer to any mixture or chemical compound that is configured to make layers of the skin softer and more pliable. Accordingly, the antiperspirant composition may include any compound or mixture that is configured to make layers of the skin softer and more pliable. Examples of emollients may include myristyl myristate.

The antiperspirant composition may include a residue masking agent. A residue masking agent may refer to any mixture or chemical compound that is configured to conceal residue of an antiperspirant composition. Accordingly, the antiperspirant composition may include any compound or mixture that is configured to conceal residue of an antiperspirant composition. An example of a residue masking agent is PPG-14 butyl ether.

The antiperspirant composition may include other ingredients such as anti-bacterial additives, dyes, antioxidants, and moisturizers among other additive to achieve a desired purpose or function. Specifically, the antiperspirant composition may include fragrances to provide the antiperspirant composition with a pleasant smell.

The antiperspirant composition may include a deodorant to reduce odors emanating from perspiration. Examples of deodorant additives include any agent to neutralize, suppress, or mask perspiration odor.

In addition to the aforementioned components, further additives may be included in the antiperspirant composition for various purposes including additives that cause the antiperspirant composition to exhibit long-lasting fragrance, odor protection, bacteria control, and/or another desired purpose and/or function. Specific examples of additional such additives include, but are not limited to, skin conditioners; pigments; preservatives; antioxidants; moisturizers; and the like.

Tables (1)-(3) present an example of an antiperspirant composition as described herein. More specifically, the antiperspirant composition may include a premix of an active antiperspirant ingredient and an active carrier in the corresponding weight percentages as indicated in Table (1).

TABLE (1)

| Ingredient | Weight Percent (%) |
|---|---|
| Aluminum Zirconium Techlorohydrex | 43.6 |
| Cyclopentasiloxane | 56.4 |

As used in Table (1), aluminum zirconium techlorohydrex may be the active antiperspirant ingredient and cyclopentasiloxane may be the active carrier of the active antiperspirant ingredient. A semi-solid antiperspirant composition may include the following ingredients in the corresponding weight percentages as indicated in Table (2).

TABLE (2)

| Ingredient | Weight Percent (%) |
|---|---|
| Active premix | 58.435 |
| Stearyl Alcohol, solid | 9.6450 |
| Soy Wax | 4.0 |
| Myristyl Myristate | 1.92 |
| PPG-14 Butyl Ether | 14.0 |
| Cetyl Alcohol | 10.0 |
| Polyethylene | 1.80 |
| Hydroxyethylcellulose | 0.1 |
| Modified Corn Starch | 0.1 |

As used in Table (2), the active premix may be the premix described in connection with Table (1). Stearyl alcohol, soy wax, cetyl alcohol, and polyethylene may be structurants, myristal myristate may be an emollient, and PPG-14 butyl ether may be a mask reducing agent. Additionally, hydroxyethylcellulose may be a non-ionic modified cellulose polymer and modified corn starch may be a non-ionic calcium salt. As described above, the active antiperspirant ingredient and the active carrier may be first mixed together as indicated in Table (1). The premix may then be combined with the ingredients indicated in Table (2) to form a semi-solid antiperspirant composition with non-ionic polymers as described herein. Table (3) indicates an overall antiperspirant composition including the active premix ingredients as indicated in Table (1) and the semi-solid ingredients as indicated in Table (2).

TABLE (3)

| Ingredient | Weight Percent (%) |
|---|---|
| Cyclopentasiloxane | 32.9573 |
| Aluminum Zirconium | 25.478 |
| Trichlorohydrex GLY | |
| Stearyl Alcohol | 9.6450 |
| PPG-14 Butyl Ether | 14.0 |
| Soy Wax | 4.0 |
| Cetyl Alcohol | 10.0 |
| Polyethylene | 1.80 |
| Myristyl Myristate | 1.92 |
| Hydroxyethylcellulose | 0.10 |
| Modified Corn Starch | 0.10 |

The antiperspirant composition as indicated in Table (3) may exhibit an average hardness of 45 grams-force when poured at 52 degrees Celsius.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant composition with non-ionic polymers, comprising:
an active antiperspirant ingredient disposed within the antiperspirant composition;
a non-ionic modified cellulose polymer disposed within the antiperspirant composition, the non-ionic modified cellulose polymer forming between 0.01 to 5.0 weight percent of the antiperspirant composition; and
a non-ionic calcium salt disposed within the antiperspirant composition, the non-ionic calcium salt forming between 0.01 to 5.0 weight percent of the antiperspirant composition.

2. The antiperspirant composition of claim 1, in which the antiperspirant composition is a semi-solid antiperspirant composition.

3. The antiperspirant composition of claim 1, in which the non-ionic modified cellulose polymer, the non-ionic calcium salt, or combinations thereof are hydrophilic film-forming ingredients.

4. The antiperspirant composition of claim 1, in which the non-ionic cellulose polymer is pseudo-plastic and is configured to retain its form in the presence of a surface stress.

5. The antiperspirant composition of claim 1, in which the non-ionic cellulose polymer, the non-ionic calcium salt, or combinations thereof, form between 0.01 to 1.0 weight percent of the antiperspirant composition.

6. The antiperspirant composition of claim 1, in which the non-ionic cellulose polymer, the non-ionic calcium salt, or combinations thereof, are configured to increase a stability of an antiperspirant composition matrix.

7. The antiperspirant composition of claim 1, in which the non-ionic modified cellulose polymer is hydroxyethylcellulose.

8. The antiperspirant composition of claim 1, in which the non-ionic calcium salt is modified corn starch.

9. The antiperspirant composition of claim 1, in which the active antiperspirant ingredient is combined with an active carrier in a premix.

10. The antiperspirant composition of claim 1, in which the antiperspirant composition further comprises an emollient, a carrier, a number of structurants, a residue masking agent, or combinations thereof.

11. An antiperspirant product with non-ionic polymers, comprising:
a container;
an antiperspirant composition housed within the container, the antiperspirant composition comprising:
an active antiperspirant ingredient;
a non-ionic modified cellulose polymer forming between 0.01 to 5.0 weight percent of the antiperspirant composition; and
a non-ionic calcium salt forming between 0.01 to 5.0 weight percent of the antiperspirant composition.

12. The antiperspirant product of claim 11, in which the antiperspirant composition is a semi-solid antiperspirant composition.

13. The antiperspirant product of claim 11, in which the non-ionic modified cellulose polymer, the non-ionic calcium salt, or combinations thereof are hydrophilic film-forming ingredients.

14. The antiperspirant product of claim 11, in which the non-ionic cellulose polymer is pseudo-plastic and is configured to retain its form in the presence of a surface stress.

15. The antiperspirant product of claim 11, in which the non-ionic modified cellulose polymer is hydroxyethylcellulose.

16. The antiperspirant product of claim 11, in which the non-ionic calcium salt is modified corn starch.

17. The antiperspirant product of claim 11, wherein the container comprises a dispenser to push the antiperspirant composition out of the container.

18. The antiperspirant product of claim 11, in which the antiperspirant composition is configured to be pushed through a grate of the container.

19. An antiperspirant composition with non-ionic polymers, comprising:
an active antiperspirant ingredient disposed within the antiperspirant composition;
hydroxyethylcellulose forming between 0.01 to 5.0 weight percent of the antiperspirant composition, the hydroxyethylcellulose configured to bind other ingredients of the antiperspirant composition; and
modified corn starch forming between 0.01 to 5.0 weight percent of the antiperspirant composition, the modified corn starch configured to bind other ingredients of the antiperspirant composition.

20. The antiperspirant product of claim 19, further comprising a dye, fragrance, or combinations thereof.
